(19) United States
(12) Patent Application Publication
(10) Pub. No.: US 2026/0249070 A1
(43) Pub. Date: Aug. 27, 2026

(54) DETERMINATION OF EJECTED VOLUME IN A PNEUMATIC PUMP SYSTEM

(71) Applicant: SYNCARDIA SYSTEMS, LLC, Tucson, AZ (US)

(72) Inventor: Frank A. Tinker, Tucson, AZ (US)

(21) Appl. No.: 19/648,050

(22) Filed: Apr. 15, 2026

Related U.S. Application Data
(63) Continuation of application No. PCT/US2024/053403, filed on Oct. 29, 2024.
(60) Provisional application No. 63/594,097, filed on Oct. 30, 2023.
(60) Provisional application No. 63/606,244, filed on Dec. 5, 2023.

(30) Foreign Application Priority Data
Oct. 29, 2024 (WO) PCT/US2024/053364

(51) Int. Cl. A61M 60/196; A61M 60/258; A61M 60/268; A61M 60/427; A61M 60/562; A61M 60/837; A61M 60/892

(52) U.S. Cl. CPC A61M 60/196; A61M 60/258; A61M 60/268; A61M 60/427; A61M 60/562; A61M 60/837; A61M 60/892; A61M 2205/04; A61M 2205/3327; A61M 2205/3334; A61M 2205/3344; A61M 2205/3368; A61M 2205/3379; A61M 2205/50; A61M 2210/125

(57) ABSTRACT

An apparatus including a two-chamber pneumatic pump system (that has its internal volume divided, by a flexible diaphragm, into an activating chamber and an activated chamber) and methodology of operating such apparatus in a manner that regularly satisfies a full-eject condition and does not require and, therefore, does not involved overcompensation of pressure of the fluid in the activating chamber—specifically, in a manner preventing the conventionally-used setting of the maximum pressure of fluid delivered to the activating chamber. Determination of a moment of time when the full-eject condition is satisfied, and of a full-eject volume.

### CROSS-REFERENCE TO RELATED APPLICATIONS

This application is a continuation of International Application No. PCT/US2024/053403 filed Oct. 29, 2024, published as WO2025/096436, which claims priority from and benefit of U.S. Provisional Application No. 63/594,097 filed Oct. 30, 2023, U.S. Provisional Application No. 63/606,244 filed Dec. 5, 2023, and International Application No. PCT/US2024/053364 filed Oct. 29, 2024. The disclosure of each of the above-identified documents is incorporated herein by reference.

### TECHNICAL FIELD

This invention is generally related to techniques for structuring and/or driving a pneumatically actuated pump system and, in particular, to an apparatus containing a pneumatic driver structured to provide pneumatic power to a pump system that has shell defining a substantially constant volume, which volume is separated by a moveable chamber separator into an activating chamber (receiving pneumatic energy from the driver) and an activated chamber receiving and releasing a fluid from and to the intended environment as a result of operation of the activating chamber.

### RELATED ART

General area for use of implementations of the idea of this invention is pneumatic pumps and, specifically, pneumatic pump systems that are air-driven and that include two chambers formed within an overall volume enclosed within the shell (or chamber housing). A chamber separator within the shell divides the shell into these two chambers. One of the chambers—referred to herein for simplicity as “activating chamber”—is connected to a power console that includes a pneumatic driver reversibly pushing a first fluid into the activating chamber (and thereby causing a reversible change of a position of the chamber separator within the overall volume of the shell and modifying the local volume of the other of the two chambers in a reversible manner). The other chamber—referred to herein for simplicity as “activated chamber”—is operably connected with local environment and is caused at least (i) to pull in from the local environment a second fluid when the movement of the chamber separator creates negative internal pressure in the activated chamber and (ii) pushes this second fluid to the local environment when the movement of the chamber separator creates positive internal pressure in the activated chamber, respectively through inlet and outlet fluidly cooperating this activated chamber with such local environment.

### Various Applications of Air-Operated Pumps

Pneumatic pumps of the variety mentioned above are commonly referred to as “air-operated diaphragm pumps”—they use an air (or, more generally—gas or even fluid) valve that directs compressed air (gas) between two sides of the pump, back and forth—and are frequently used to transfer liquids with a broad range of viscosities as well as liquids with suspended solids. They are used in facilities of all sizes, and in a variety of different industries—from petrochemical to food and beverage, these pumps are popular and versatile. They also can handle many aggressive chemicals such as acids because they can be constructed with a wide variety body materials and diaphragms. In particular, conventional designs lend themselves to transferring highly abrasive or viscous products. See, for example, blog.craneengineering.net/how-do-air-operated-double-diaphragm-pumps-work. See also www.arozone.com/en-us/technologies/how-diaphragm-pumps-work. As general workhorse devices, such pneumatic pumps are utilized in practically every known area unrelated to the management and care of a patent, such as in:
Mining—for dewatering above and below ground sites and quarries, transferring of slurries and sludge, transferring of fuel for vehicles or heavy construction equipment;
Coatings—for transferring, dispensing and dosing paint or ink, feed pump for spray guns;
Ceramics industries—for ceramic slips, day tank transfer, filling molds;
Chemical Processing—for loading and unloading tankers, portable utility pump, batching or dosing chemicals;
Electroplating and Anodizing—for replenishing chemicals in plating or metal finishing tanks, agitation of cleaning solutions, filtration;
Pulp and Paper industries—for printing inks, glues, bulk adhesive transfer;
Oil and Gas industries—for filter press, bulk fuel transfer, settling pond transfer;
Water and Wastewater Treatment—for transferring suspended solid media, filter press, utility pump, to name just a few.

At the same time, pneumatic pumps of the air-operated diaphragm version have also been employed in one rather specific—but not limiting in any shape or form—application related to an artificial heart system. To this end, the reader may benefit from some excursion into the structure and mechanics of operation of the heart muscle and related solutions—which are touched upon below.

The heart is the muscle that drives the cardiovascular system in living beings. Acting as a pump, the heart moves blood throughout the body to provide oxygen, nutrients, hormones, and to remove waste products. The blood follows two separate pathways in the human body, the so-called pulmonary and systemic circulatory circuits. In the pulmonary circuit, the heart pumps blood first to the lungs to release carbon dioxide and bind oxygen, and then back to the heart. Thus, oxygenated blood is constantly being supplied to the heart. In the systemic circuit, the longer of the two, the heart pumps oxygenated blood through the rest of the body to supply oxygen and remove carbon dioxide, the byproduct of metabolic functions carried out throughout the body. The heart supplies blood to the two circuits with pulses generated by the orderly muscular contraction of its walls.

In order to keep blood moving through these two separate circulatory circuits, the human heart has four distinct chambers that work in pairs. As illustrated in FIG. 1, the heart 100 includes a right atrium 112, a right ventricle 114, a left atrium 116, and a left ventricle 118. One pair of chambers, the right ventricle and left atrium, is connected directly to the pulmonary circuit. In it, de-oxygenated blood from the body is pumped from the right ventricle 114 to the lungs, where it is oxygenated, and then back to the left atrium 116.

In the systemic circuit, the other pair of chambers pumps the oxygenated blood through body organs, tissues, and bones. The blood moves from the left atrium 116, where it flows from the lungs, to the left ventricle 118, which in turn pumps the blood throughout the body and all the way back to the right atrium 112. The blood then moves to the right ventricle 114 where the cycle is repeated. In each circuit, the blood enters the heart through an atrium and leaves the heart through a ventricle.

Thus, the ventricles 114, 118 are essentially two separate pumps that work together to move the blood through the two circulatory circuits. Four check valves control the flow of blood within the heart and prevent flow in the wrong direction. A tricuspid valve 120 controls the blood flowing from the right atrium 112 into the right ventricle 114. Similarly, a bicuspid valve 122 controls the blood flowing from the left atrium 116 into the left ventricle 118.

Two semilunar valves (pulmonary 124 and aortic 126) control the blood flow leaving the heart toward the pulmonary and systemic circuits, respectively. Thus, in each complete cycle, the blood is pumped by the right ventricle 114 through the pulmonary semilunar valve 124 to the lungs and back to the left atrium 116. The blood then flows through the bicuspid valve 122 to the left ventricle 118, which in turn pumps it through the aortic semilunar valve 126 throughout the body and back to the right atrium 112. Finally, the blood flows back to the right ventricle 114 through the tricuspid valve 120 and the cycle is repeated. When the heart muscle squeezes each ventricle, it acts as a pump that exerts pressure on the blood, thereby pushing it out of the heart and through the body. The blood pressure, an indicator of heart function, is measured when the heart muscle contracts as well as when it relaxes. The so-called systolic pressure is the maximum pressure exerted by the blood on the arterial walls when the left ventricle of the heart contracts forcing blood through the arteries in the systemic circulatory circuit. The so-called diastolic pressure is the lowest pressure on the blood vessel walls when the left ventricle relaxes and refills with blood. Healthy blood pressure is considered to be about 120 millimeters of mercury systolic and 80 millimeters of mercury diastolic (usually presented as 120/80).

Inasmuch as the function of the circulatory system is to service the biological needs of all body tissues (i.e., to transport nutrients to the tissues, transport waste products away, distribute hormones from one part of the body to another, and, in general, to maintain an appropriate environment for optimal function and survival of tissue cells), the rate at which blood is circulated by the heart is a critical aspect of its function. The human body has a built-in mechanism (the so-called Frank-Starling mechanism) that allows it to increase the filling pressure at the heart automatically based on the oxygen requirements of the body. This, coupled with the autonomic increase in heart rate and ejection fraction, allows the circulatory system to deliver blood at a rate necessary to meet the persons' need. Such flow rate is known as ‘cardiac output’. The cardiac output in a healthy human body may vary from about 4 to about 15 liters per minute (LPM), according to the activity being undertaken by the person, at a heart rate that can vary from about 50 to about 180 beats per minute.

Several artificial devices have been developed over the years to supplement or replace the function of a failing heart in patients. These include devices developed by companies as well as research institutions such as the Berlin Heart Institute, the Pennsylvania State University, the University of Utah, the Cleveland Clinic Foundation, the University of Perkinje (in Bruno, Czechoslovakia), the University of Tokyo, the Thoratec Corporation, Abiomed Inc., Novacor, and Symbion Inc. Typically, these artificial devices include pumps that aim at duplicating the required pumping functions of the left and right human ventricles. One method of actuation for these pumps has been through the pneumatic action of an external mechanism. See, for example, U.S. Pat. Nos. 4,611,578 and 5,766,207. Periodic pulses of compressed air drive the pumps at the desired pressure and rate of cardiac output. A moderate vacuum may be applied between pulses to allow more rapid refilling of the ventricles with blood flowing from the respective atrium.

(As an aside, one notable air-actuated diaphragm pump configured as an artificial heart system is currently in use as an implant for patients waiting for a heart transplant is the Total Artificial Heart manufactured by SynCardia Systems, LLC of Tucson, Arizona. Designed to operate much the same way as a human heart, this pump system replaces the two active chambers (i.e., the ventricles) of the actual human heart with corresponding artificial hardware components. As illustrated in FIG. 2, an embodiment of such air-actuated pump system 200 includes two separate chambers (sometimes interchangeably referred to as ventricles) 212 and 214 that replace, in practice, the right and left natural ventricles of the human heart, respectively. Each chamber is equipped with a respective diaphragm (schematically indicted as 216 and 218 in the right and left chamber, respectively) that has an air contact side and a blood contact side. Each diaphragm may be designed to have a shape, in an unloaded/rest position, substantially similar to that of as a spherical hemisphere. The bottom of each chamber is provided with an airline (240 and 242, in the right and left chamber, respectively). Each of the chambers is equipped with judiciously designed valves: artificial valves 244A (which may be referred as “tricuspid” valve of the pump system 200), 246A (“bicuspid” valve of the pump system 200) and 244B (“pulmonary” valve of the pump system 200), 246B (“aortic” valve of the pump system 200).

As shown in FIG. 3, in practice and prior to operation, the artificial-heart-like air-actuated pump system 200 is implanted by connecting the top of the right chamber 212 to the right atrium 112 of the human body and the top of the left chamber 214 to the left atrium 116 of the human body. The airlines 240, 242 are at least partially embedded in the patient's body and are connected to a pneumatic driver (in some currently implemented cases, the airlines 240, 242 extend outside for connection to the external-to-the-body pneumatic driver).

When driven by a supply of pressurized air from the pneumatic driver, each diaphragm 216, 218 discharges blood from the respective chamber 212, 214—thereby simulating the function of a natural ventricle. This phase is referred to in the art as systole or equivalently as the ejection phase. When the pressurized air is removed from the diaphragm (during a portion of the cycle known as diastole or the filling phase), blood can enter the ventricle from the connected atrium. The rate at which blood enters the ventricle depends on the difference between the atrial pressure and the pressure on the air-side of the diaphragm. To increase this filling rate, a slight vacuum of about 10 mm Hg or so is normally applied to the air-side of the diaphragm during diastole. The artificial valves 244A, 246A, 244B, and 246B are configured to control the flow from the respective atrium into each artificial ventricle and out to the circulatory systems, respectively. Understandably, one of the pump actuators 212, 214 uses appropriate valves to direct blood through the lungs (pulmonary system) of the human subject, while the tother pumps blood through the systemic system (the body).

The pneumatic drivers used to date still somewhat limit the degree of independent mobility of the patient. Such drivers employ compressors, vacuum pumps, and air tanks coupled to electrically actuated valves, all of which amounts to a bulky apparatus that needs to be attended to and requires to be transported by and/or with the patient. Even at the approximate weight of 15 pounds and size of about 0.3 cubic feet achieved so far (the example of which is provided by the Freedom driver available from SynCardia Systems, LLC), pneumatic drivers remain an obstacle to artificial heart patients' quality of life. It is essential that the pneumatic driver be able to provide the correct pressure balance between the left and right ventricles of the artificial heart to ensure the proper operating pressure to the pulmonary and systemic circuits regardless of the speed of operation. Typically, this requires that the driver be able to operate so as maintain, on average a mean pulmonary artery pressure of about 35 mmHg and a mean aortic pressure of about 95 mmHg. To this end, sufficient fill volume and heart rate must be achieved to supply a minimum cardiac output of 3.5 LPM.

Substantially in every application, the operation of an air-driven (more generally—fluidically-driven, as the skilled person will appreciate) pump system such as a pump with an air-driven diaphragm remains inefficient. The loss of efficiency is caused—at least in part—by operating a pump system that contain pneumatically driven activating and activated chambers (separated from one another by a flexible diaphragm) without paying attention to whether and/or when the second fluid present in the activated chamber is, under the action of the pressurized first fluid in the activating chamber, entirely forced out by the end of the pump cycle. (Such end state may be referred to as full-eject of the pump contents, or satisfaction of the full-eject condition.)

### SUMMARY OF THE INVENTION

Embodiments of the invention provide an apparatus that includes a pump system having a shell defining an internal volume that is separated by a flexible and reversibly movable diaphragm into an activating chamber and an activated chamber, a pneumatic driver configured to provide pneumatic power to the pump system and containing a compression mechanism and a gauge apparatus cooperated with activating fluid provided to the activating chamber by the compression mechanism, and electronic circuitry. The apparatus is configured to satisfy the full-eject condition during the operation of the pump system. The compression mechanism is configured to reversibly reposition an activating fluid between a first fluid location and the activating chamber/ The gauge apparatus is configured to measure at least a time-dependent value of pressure produced by the activating fluid at the activating chamber during an operation of the pump system and one or more of (i) a time-dependent value of a change in a volume of the compression mechanism; and (ii) a number of moles of the activating fluid delivered to the activating chamber during a systolic portion of a pump cycle and temperature at the activating chamber, and then to generate data representing results of such measurement. The electronic circuitry is configured (a) to determine in real time and based at least in part on the data received from the gauge apparatus: a moment of time when an operation of the pump system satisfies a full-eject condition, and a change of a volume of an activated fluid in the activated chamber that occurs between a beginning of the systolic portion of a pump cycle of the pump system and such moment of time. The electronic circuitry is further configured to produce a user perceivable output that represents a result of such determination. In at least one embodiment of the apparatus, the compression mechanism may include a piston repositionable in a piston housing, and the gauge apparatus may be configured to identify, as a function of time, a position of the piston within the piston housing; or the compression mechanism may include a source of compressed activating fluid while the gauge apparatus includes a mass-flow sensor gauge and a device configured to measure temperature (such as a thermometer containing device). Alternatively or in addition, and substantially in every implementation of the apparatus, at least one of the following conditions can be satisfied: (i) the chamber separator is configured as a flexible diaphragm that is substantially fluidly impenetrable; (ii) a portion of the compression mechanism containing the activating fluid is fluidly connected only with the activating chamber; and (iii) the electronic circuitry is configured to determine, as a function of time and based on said data, a value of a time derivative of the at least time-dependent value of pressure produced by the activating fluid at the activating chamber. Optionally, substantially every embodiment of the apparatus may be configured to satisfy one or more of the following: (a) the electronic circuitry is structured to activate a vent valve cooperated with a driveline that fluidly connects the activating chamber with the pneumatic driver to release pressure from the driveline to initiate a diastolic portion of the pump cycle substantially at the moment of time when the full-eject occurs, and (b) the electronic circuitry is configured as a processor programmed to cause the pneumatic driver to reverse a flow of the activating fluid between the first location and the activating chamber substantially at such moment of time. Optionally, a) the compression mechanism includes a spatially-repositionable component or b)—the compression mechanism incorporates at least one piston slidably mounted inside a piston housing and/or a source of compressed fluid, and/or—the compressing mechanism is configured to reposition the activating fluid from the first fluid location to the activating chamber. Alternatively or in addition, and substantially in every implementation of the apparatus, the activated chamber may be structured to include an inlet and an outlet spatially separated from one another while the activating chamber may be structured to include a single aperture fluidly connected with the compression mechanism. Optionally—and at least in one embodiment—the electronic circuitry may be configured to determine whether the pump system satisfies the full-eject condition by determining a time-dependent value of a time-derivative of a function of at least the time-dependent value of the pressure. (In the specific case of the latter, the function may be defined as (i) a product of a volume of the activating fluid in the compression mechanism being repositioned between the first fluid location and the activating chamber and the pressure or as (ii) a natural logarithm of such product.) Furthermore, substantially every implementation of the apparatus may include a tangible non-transitory computer readable medium, operably coupled with the electronic circuitry and containing program code for determining the moment of time when the full-eject condition is satisfied by solving an equation in which a time-derivative of a sum of (i) a function of a reading of the mass-flow sensor gauge connected to the activating chamber, (ii) the function of the pressure, and (iii) a negative of the function of the temperature is substantially equal to zero, and/or program code for determining such moment of time by solving an equation in which a time-derivative of a sum of (a) the function of a reading of a piston position gauge of the gauge apparatus and the function of the pressure is substantially equal to zero. (In the special case of the latter, the function may be a natural logarithm.) Optionally, the pump system may be configured as a ventricle portion of an implantable artificial heart apparatus.

Embodiments further provide for use of an embodiment of apparatus alluded to above for repositioning the chamber separator configured as a flexible substantially fluidly-impenetrable diaphragm within the rigid shell to change at least one of a volume of the activating chamber and a volume of the activated chamber and/or for exchanging blood between an implantable artificial heart apparatus and a pulmonary artery and/or an aorta.

Embodiments additionally provide a computer-implemented method that includes carrying out the following steps with the use of a computer: (a) recording, as a function of time, at least a value of pressure produced by the activating fluid in the activating chamber of the pump system of substantially every embodiment of the apparatus alluded to above during repositioning of the activating fluid between the compression mechanism and the activating chamber of the pump system and measured with the gauge apparatus; (b) identifying, as a function of time, at least one of (i) a temperature of the activating fluid, a parameter related to a volume occupied by the activating fluid, a mass flow rate of the activating fluid between the first fluid location and the activating chamber, and (ii) a position of a spatially-repositionable component of the compressing mechanism of the apparatus. Optionally, such function may be defined as a time derivative of the at least said value of pressure or a time-derivative of a natural logarithm of the at least said value of pressure. These steps are performed to characterize an operation of the apparatus. Optionally, and embodiment of such method may additionally include defining—with the use of the computer and with the use of results of the recording and identifying—at least a moment of time when the pump system satisfies a full-eject condition during operation thereof. Alternatively or in addition, and substantially every implementation of such method may include determining, with the use of the computer, a full-eject volume characterizing the operation of the pump system. Optionally,

[symbol]

Embodiments additionally provide A computer program product for governing an operation of a pneumatically driven pump apparatus configured to satisfy a full-eject condition during the operation thereof. The computer program product includes a computer usable tangible non-transitory medium having computer readable program code thereon, where the computer readable program may include (a) program code for determining a moment of time when a pump system of the apparatus, operably fluidly connected with a pneumatic driver of the apparatus, satisfies the full-eject condition; and (b) program code for ascertaining a full-eject volume characterizing the operation of the pump system. In at least one implementation of the computer program product, the program code for determining includes program code for determining the moment of time based on a product of a value of pressure of activating fluid delivered from the pneumatic driver to the activating chamber of the pump system and a value representing a volume of the activating fluid repositioned by the pneumatic driver to the activating chamber. At least in one embodiment of the program product, the program code for ascertaining may include (i) program coder for defining a sum of a first function of a reading of a piston position gauge of a gauge apparatus of the pneumatically drive pump apparatus and a second function of a pressure of activating fluid produced by the pneumatic driver at the activating chamber, and/or (ii) program code for defining a sum of a third function of a reading of a mass-flow sensor gauge of the gauge apparatus, a fourth function of a temperature at the activating chamber, and said second function.

Embodiments further provide a method, according to which operating substantially every embodiment of the apparatus alluded to above is carried out by at least (i) measuring a moment of time when the operation of the pump system satisfies the full-eject condition, and (ii) with the use of the electronic circuitry performing one of the following: (a) when the compression mechanism includes a source of compressed fluid, activating a vent valve cooperated with a driveline that fluidly connects the activating chamber with the pneumatic driver to release pressure from the driveline to initiate a diastolic portion of the pump cycle substantially at such moment of time; and (b) when the compression mechanism includes a piston repositionable within a piston housing, causing the pneumatic driver to reverse a flow of the activating fluid between the first location and the activating chamber substantially at such moment of time. Optionally, such method may additionally include maintaining fluidic pressure provided by the pneumatic driver to the activating chamber during a systolic portion of a pump cycle to maintain a full-eject volume from an activated chamber of the pump substantially constant from one pump cycle to another pump cycle.

### BRIEF DESCRIPTION OF THE DRAWINGS

The invention will be more fully understood by referring to the following Detailed Description of Specific Embodiments in conjunction with the Drawings, of which:

FIG. 1 is a schematic representation of a human heart.

FIG. 2 is a schematic view of an embodiment of the SynCardia LLC artificial heart apparatus.

FIG. 3 is a representation of the artificial heart of FIG. 2 connected to the heart atria of a human body.

FIG. 4 is a schematic representation of a portion of an embodiment of the invention/

FIGS. 5A, 5B illustrate, respectively, situations when the main chamber of the pump-system of the embodiment of FIG. 4 in a full-fill status and in a full-eject status.

FIG. 6: Schematic of operation of a volume-changer type drive with the pneumatic pump-system of FIG. 4.

FIG. 7A: Schematic of operation of the compressed air-source type drive with the pneumatic pump-system of FIG. 4 during the phase of filling the activating chamber of the pump-system with gas (~ the systolic portion of the pump cycle).

FIG. 7B: Schematic of operation of the compressed air-source drive with the pneumatic pump-system of FIG. 4 during the phase of filling the activated chamber of the pump-system with external fluid (~ the diastolic portion of the pump cycle).

FIGS. 8A, 8B are plots (waveforms) representing, as functions of time, pressure (FIG. 8A) of the activating fluid (air, in one implementation) at and outflow of the activating fluid from (FIG. 8B) the activating chamber of the pump of FIG. 4 during the pump cycle.

FIG. 9 presents the results of measurements of pump cycles of two different embodiments of the pump, performed according to an embodiment of the detection scheme configured according to the idea of the invention.

FIGS. 10A, 10B illustration of experimental data acquired during the process of determination of whether a given embodiment of the pneumatically-driven pump apparatus satisfies the full-eject condition performed with the use of an embodiment of the invention.

FIG. 11 schematically illustrates an embodiment of a pneumatically-driven pump apparatus structured according to the idea of the invention and equipped with a pneumatic driver containing a volume-change type compression mechanism.

FIG. 12 schematically illustrates an embodiment of a pneumatically-driven pump apparatus structured according to the idea of the invention and equipped with a pneumatic driver containing a source of compressed fluid (gas/air).

FIGS. 13A, 13B present typical empirical data illustrating the correspondence between values of the measured full-eject volume of the fluid out of the outlet of the activated chamber of an embodiment of the pump system and those computed for air-operated pump system for two different sizes of the pump system (50 cubic centimeters and 70 cubic centimeters) using multiple pneumatic actuators under a wide variety of conditions.

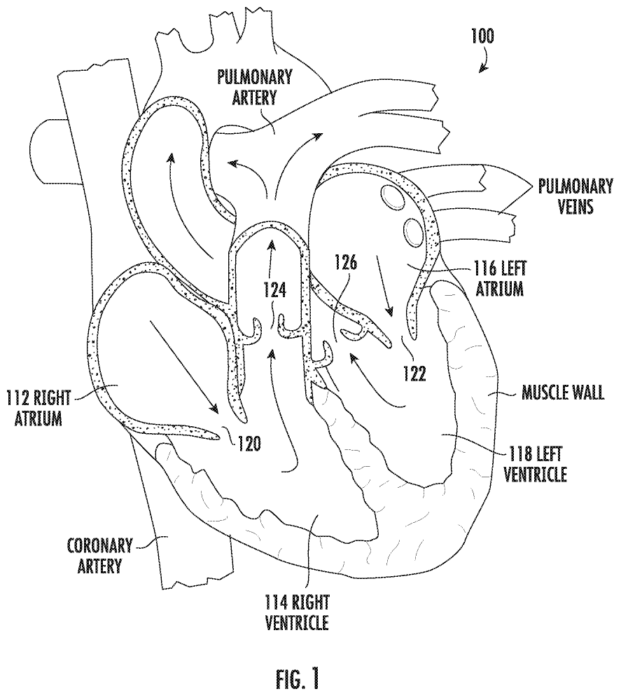

### DETAILED DESCRIPTION

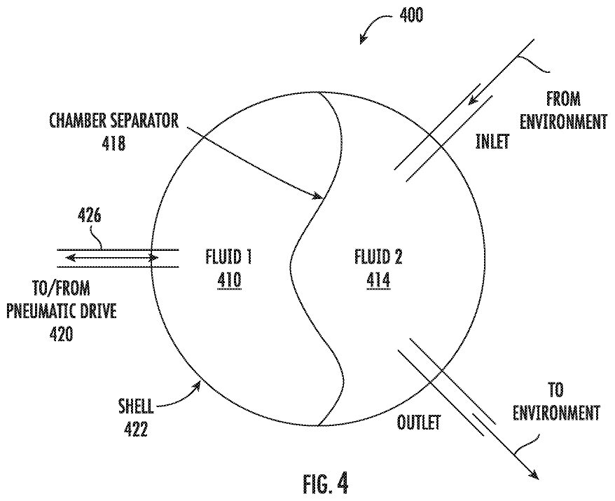

FIG. 4 schematically illustrates a portion of the fluid-driven (in one case—air driven) pump system 400 of interest. Here, fluid 1 (such as air or gas, in one example, reversibly filling the activating chamber 410 that is separated from the activated chamber 414 by the reversibly moveable chamber separator 418; shown in this example as a flexible diaphragm) is pumped in and out of the chamber 410 by the pneumatic drive 420 (not shown), thereby reversibly pushing the flexible chamber separator 418 towards the wall of the chamber 414 and causing, in alternation, at least partial emptying of the local volume of the chamber 414 through the outlet and at least partial filling of this local volume through the inlet. As fluid 1 is removed from the activating chamber 414, fluid 2 can enter the activated chamber 414 and vice versa. The pump 400 may be operated with, for example, a piston/cylinder driver connected to the pump system to alternatively reduce and then expand the volume of the activating chamber, or be of a valve-operated type known in the art.

For proper operation, inlet/outlet of the activated chamber 412 are equipped with respective appropriately structured valves, as a person of ordinary skill in the art would readily understand.) The chamber separator is judiciously configured such that, depending on its position/orientation within the overall volume defined by the shell 422, either of the corresponding local volumes of the chambers 410, 414 can be reduced to substantially zero or increased to substantially coincide with the overall volume. For example, in the case when the inner surface of the shell 422 defines a substantially spherical surface, the chamber separator 418 may be designed to have a shape, in an unloaded/rest position, substantially similar to that of as a spherical hemisphere (in one case, to be configured as a flexible diaphragm). The shell is configured to substantially not allow the overall volume within the shell to be varied, for example the shell may be made from a mechanically rigid material such as isoplast or stainless steel. Understandably, the chamber separator 418 can be, generally, a piston, a membrane or diaphragm, or other moveable or distensible means to keep the fluid in the activated chamber and the air in the activating chamber separated while the latter forces the former from the outlet of the activated chamber. Appreciably, substantially any complementary shapes of the shell 422 and the chamber separator 418 can serve provided the means for keeping the first and second fluids separate will, under the action of the pressurized first fluid, fully distend or move into a position such that there is no longer any room in the shell profile for the second fluid that needs to be pumped.

In describing various embodiments of the apparatus of the invention and/or methodology of the invention, the pump system may be interchangeably referred to as air-operated (or driven), gas operated (or driven), or, more generally, fluid-operated (or driven) or by a similar term. Each of such references or descriptions implies that the chamber separator 418 is moved or repositioned in response to pressure of fluid/air/gas formed by the pneumatic drive 420 on the activating-chamber side of the pump system.

When using an air-actuated pump such as that referred to above, it is frequently necessary to ascertain if (or whether) the fluid in the activated chamber at the beginning of the pump cycle has been entirely forced out of the pump by the end of the pump cycle due to operation of the chamber separator driven by the pressurized fluid (for example, air) delivered into the activating chamber—that is, whether the full-eject condition has been satisfied. In pump systems where there is no in situ mechanism to measure the fluid contents of the pump and, therefore, it is of value in these pump systems to identify if full-eject has taken place. Accordingly, in accordance with embodiments of the present invention, methods and apparatus are disclosed for identification/determining of whether the activated chamber of a multiple-chamber fluid pump system, driven by an activating chamber connected to a pneumatic drive, has all fluid content of the activated chamber fully ejected from it.

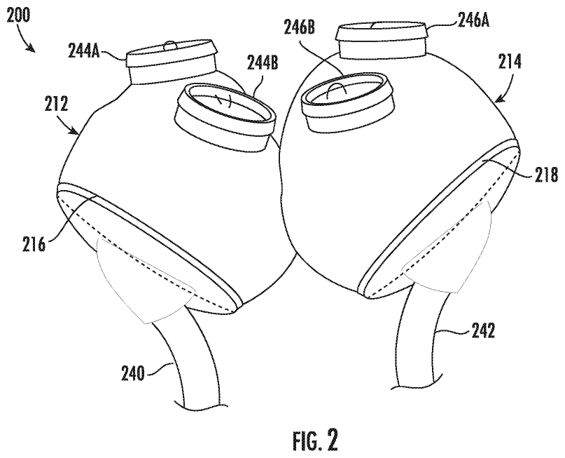

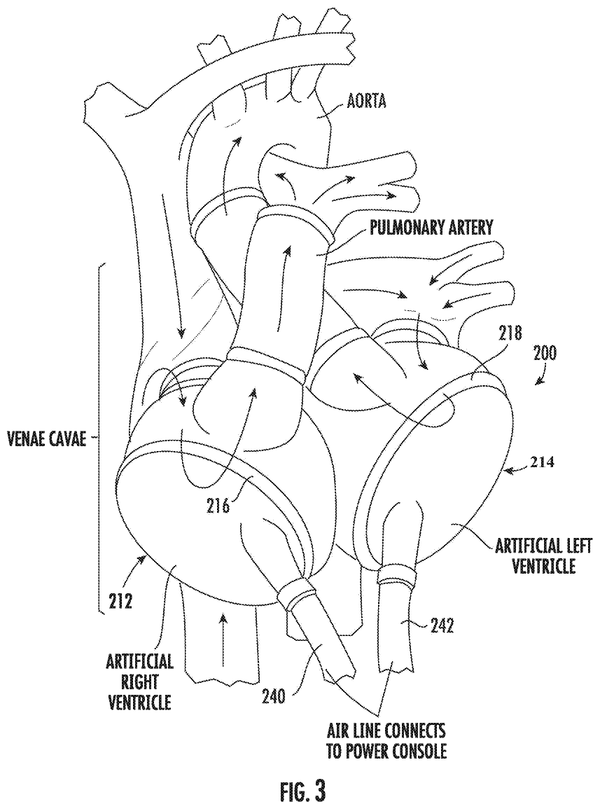

Explanations of some aspect(s) of embodiments of the invention may be presented below in reference to a very specific and non-limiting example when the discussed pump system is used in an artificial heart apparatus. However, as a skilled artisan readily understands, such reference is used only as an example while plethora of other, non-medical applications of the discussed pump system exist, most of which involve non-animate situations. See, for example, blog.craneengineering.net/how-do-air-operated-double-diaphragm-pumps-work. In such specific non-limiting example, when the pump system 400 is used as a ventricle of the artificial heart such as ventricle 212 of FIG. 2, for example, the activating chamber 410 may be an air chamber—that is, the first fluid is air, the activated chamber 414 is a blood chamber—that is, the second fluid is blood, and the pipe or passage 426 represents the external pneumatic access to the chamber 414, similarly to the airline of the embodiment of FIG. 2, the inlet is a blood chamber inlet, and the outlet is a blood chamber outlet, while the flexible membrane 418 separated blood from air/gas.

By analogy with terminology used describe a cardiac cycle, a portion of the overall pumping cycle (the cycle of operation of the pump system 400) during which contents of the activated chamber 412 are being driven out (ejected) from the activated chamber by repositioning of the chamber separator 418 (caused by increasing pressure inside the activating chamber 410) may be referred to in this disclosure as a systolic portion of the pump cycle. Similarly, the remaining portion of the overall pumping cycle during which the activated chamber 412 is being filled with contents through its inlet driven by a reversed repositioning of the chamber separator 418 (which is now moving to reduce the volume of the activating chamber) may be referred to as a diastolic portion of the pump cycle.

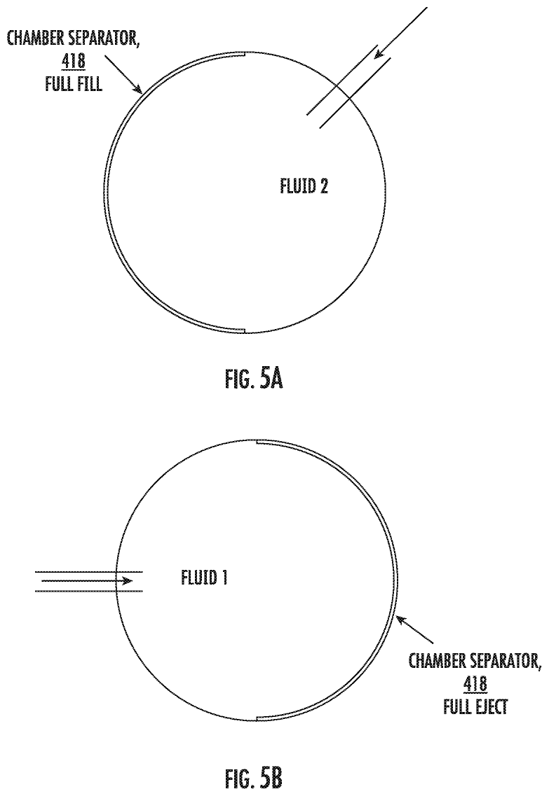

With reference to the schematics of FIGS. 5A, 5B, when fluid 1 in the chamber 410 substantially completely fills the overall volume of the volume-limiting shell or housing 422 during the diastolic portion of the pump cycle (this corresponds to the situation when the local volume of the activated chamber 414 is reduced substantially to nothing), the condition known as full-fill is satisfied. Alternatively, when fluid 1 in the activating chamber 410 substantially completely fills the overall volume of the shell 422 during the systolic portion of the pump cycle (which corresponds to the situation when the local volume of the activating chamber is reduced to practically nothing), the condition known as full-eject is satisfied.

Why is the question of whether full-eject occurs is even of importance?—The need to be able to confirm that the full-eject condition has been, indeed satisfied is driven by persisting shortcomings and problems caused in operation of a typical air-driven pump when this is not known.

In particular—as is well appreciated in related art—one approach presently used to ensure that the full-eject takes place is to set the maximum pressure of fluid 1 (that is reached during the portion of the pneumatic drive operation cycle delivering fluid 1 to the activating chamber 410) to a value that is greater than operationally required. In other words—to inject enough air into the activating chamber 410 at a high enough pressure that it is physically impossible for any liquid to avoid being ejected from the activated chamber 412 of the pump. At the end of the expulsion phase, the pressure in the pump will be equal to the maximum pressure designed into the actuator system. Therefore, the process of determining that full-eject takes place is simplified to simply assuring that that maximum pressure is achieved at the end of the expulsion phase of the pump cycle.

(Only for the sake of entertaining a specific example of an artificial heart system, this corresponds to setting the maximum pressure of air/gas delivered through the connecting pipe 426 with the use of pneumatic drive during systole to a value greater than what is seen as clinically necessary for full-eject. In such specific example, the currently conventionally used approach is to ensure that the maximum pressure is simply set to a value above that ever expected to be encountered: automated monitoring of full-eject is not typically implemented in artificial heart driver design; rather, conventional systems of related art simply allow an operator setting of the maximum driver pressure or the drive pressure to be set to a high enough value to properly perform in worst-case situations.)

This status quo “overshooting” operation of the pump system causes multiple shortcomings and/or problems. For example, in a pneumatically activated device, the more pressurized gas (fluid 1) is added to the activated chamber 410 of the embodiment, the greater is energy required to fully eject fluid 2 from the chamber 414. Therefore, knowledge of the very fact of occurrence (and/or the moment of occurrence) of the full-eject allows the user to end the current “fluid 2 pump-out” phase of the operation of the system 200 and to begin a new “fluid 2 pump-in” phase resulting in minimization of energy required to activate the system 200.

Furthermore (and in the case of use of the pump 400 in the artificial heart system, for example), since the health of blood (and, therefore, the ability of blood to perform its functions such as transporting oxygen and nutrients, forming blood clots to prevent excess blood loss etc.) may be and practically often is sensitive to the presence of and contact with artificial materials, the damage to blood passing through the chamber 414 may manifest and be more pronounced the longer the blood is in contact with artificial surfaces of the chamber 414. Here, it would be beneficial to minimize dwell time of blood in a ventricle of the artificial heart composed of synthetic materials. Therefore, knowledge that all blood has left the ventricle prior to the end of a cycle is pertinent to ensuring the minimum dwell time.

In addition, in the same specific case, the longer is the time during which air/gas is being injected into an artificial ventricle the lower is the rate at which such ventricle can be operated. Therefore, the addition of air/gas to the air/gas side of the ventricle after full-eject has taken place results in a reduced heart rate and, thereby, a reduced maximum cardiac output.

For any these reasons, the very knowledge of if (and, preferably, when) full-eject has occurred results in a safer, more efficient, and more capable driver mechanism of the discussed pump system.

According to the idea of the present invention, a persisting in related art problem of lack of knowledge of whether the pump system (that includes a pneumatically activating chamber and an activated chamber separated from one another by a chamber separator such as, for example, a flexible diaphragm) reaches the full-eject condition is solved by observing the change of a derivative of a pressure in the activated chamber of the pump system as a function of time to determine whether during the operation of a given pump the full-eject occurs (thereby immediately differentiating those pumps that operate as desired from those possibly considered to be deficient) and, based on results of such determination, making a decision of whether such given pump is required to be operated with a different pneumatic driver in order to reach the full-eject during operation.

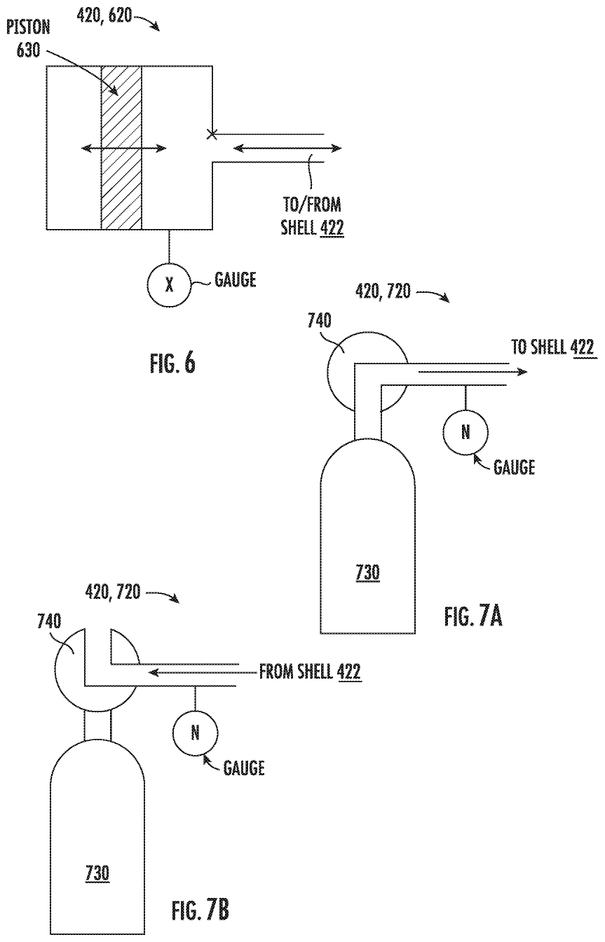

Examples of Types of Drivers. Although the general approach discussed above is applicable to substantially all drivers that may be used with the pump system 400, there are two general classes of pneumatic drivers currently commonly in use. The first class employs a compressing mechanism utilizing a piston/cylinder arrangement that is connected directly to the activating chamber of the pump-system and that maintains a substantially fixed number of molecules of fluid 1 in the combined driver/activating chamber volume throughout a phase of the cycle during which fluid 1 is delivered/pumped into the activating chamber. This class of compressing mechanism (pneumatic driver) 420, often referred to as a volume-change device, is schematically depicted in FIG. 6 as 620. Another class of drivers utilizes a source of compressed fluid 1 (air/gas) 730 (such as a high-flow compressor, compressed air tank, etc.) and appropriate pneumatic valves (not shown, in line 426) to introduce such compressed fluid (air/gas) into the activating chamber side of the pump system 400, and another pneumatic valve 740 to allow air/gas to be expelled from the activating chamber 410 of the shell 422. In some instances of using a driver of this class, a vacuum pump can be additionally used to aid the rate at which fluid 1 (air/gas) can exit the shell 422. The operation of this class of pneumatic drive 420 is schematically illustrated in FIGS. 7A, 7B as operation of the drive 720.)

### EXAMPLE 1

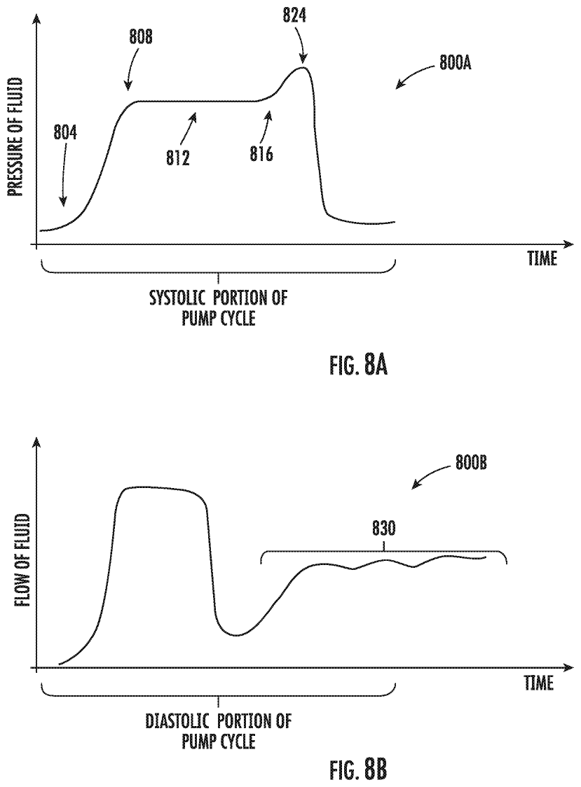

With that said, one implementation of the invention is rooted in the empirical observation and/or detection of a typical time-dependencies of waveforms characterizing air pressure (i.e., the fluid 1 pressure) at the activating chamber 410) during the pumping process. Shown in FIGS. 8A, 8B, respectively, these waveforms result from the pumping action itself. In reference to the arrangement of FIG. 4, for example, the waveform of FIG. 8A represents the typical pressure of fluid 1 delivered into the activating chamber 410 when fluid 2 is being ejected out of the outlet of the activated chamber 412 (the systolic portion of the pump cycle), while the waveform of FIG. 8B represents the flow of the fluid 1 flow out of the activating chamber 410 when fluid 2 enters the activated chamber through the inlet of the activated chamber or, alternatively, the flow of fluid 2 itself into the activated chamber (a diastolic portion of the pump cycle).

Notably, the transition of the waveform from the waveform 800A of FIG. 8A to 800B of FIG. 8B follows an informative pattern. The leftmost portion 804 of the curve 800A corresponds to the time window when the air (~ fluid 1) is just beginning to enter the activating chamber 410, so the pressure inside the activating chamber begins to rise At the beginning, pressure produced by fluid 1 increases at the beginning of the operation of the compression mechanism (initial reduction of the piston/cylinder volume, in case of utilizing pneumatic driver 620), leading to the fluid 2 being pumped out of the activated chamber at about 808. A portion 812 of the curve (between 804 and 808) corresponds to the continued operation of the compression mechanism, when fluid 2 ejected from the activated chamber increases the available volume of the activating chamber to substantially match the reduction in the piston/cylinder volume (in case of utilizing pneumatic driver 620). This flattening of the waveform 800A in the range 812 indicates that the amount of pressurized air (fluid 1) entering the activating chamber 410 is substantially matched by the amount of fluid 2 being pumped out of the activated chamber through the outlet into the environment fluidly connected with the pump: no net volume change occurs, thus this fluid volume-matching keeps the pressure during this portion of the pump cycle and in this region 812 of the curve 800A relatively constant. At the end of this flattened portion 812 of the curve 800A, at 816, substantially no more fluid 2 remains in the activated chamber and the additional air (fluid 1) being forced into the activating chamber 410 of the pump causes the pressure to abruptly rise. The abrupt peaking 824 of pressure in the activating chamber occurs because the contents of the activated chamber have been fully expelled. That is, the peaking behavior at the end of the fluid 2 expulsion phase of the pump cycle indicates that full-eject has occurred. This feature of the waveform 800A may be referred to as a “full-eject feature” of the pressure in the activating chamber and its detection during the pumping cycle) serves as indicia that full-eject has taken place.

The flattened peak of the curve represents a period of decompression, while part 830 of the waveform 800B represents the portion of the pump cycle when fluid 2, being driven through the inlet into the activated chamber 412 of the shell by the reverse repositioning of the chamber separator 418, substantially fills the activated chamber 412 and when fluid 1 is leaving the activating chamber 410 due to displacement caused by the in-flow of the fluid 2 into the activated chamber of the shell.

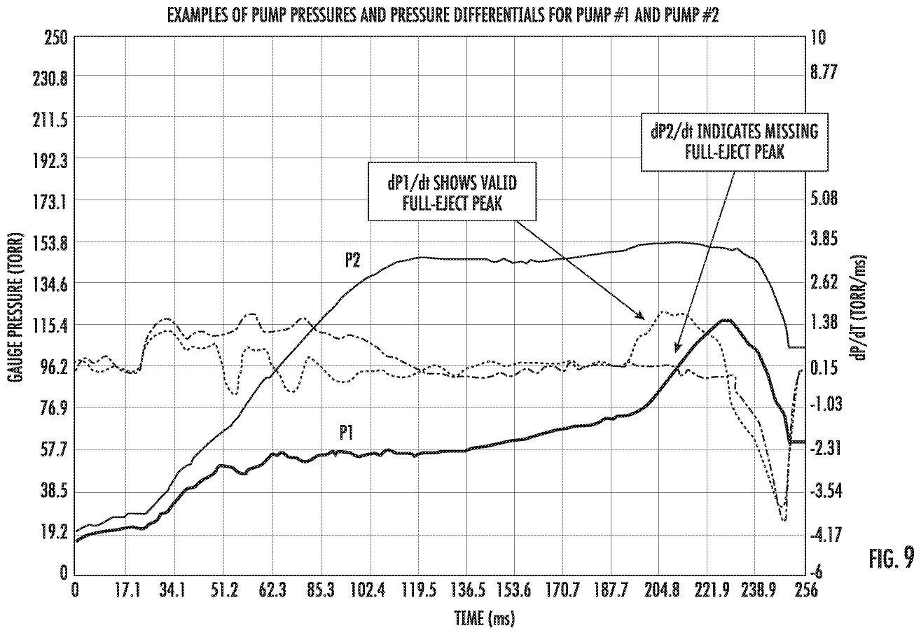

Accordingly, in one implementation of the idea of the invention, the observation or detection of the change in time-derivative of pressure at the activating chamber 410 serves to identify the beginning portion of the full-eject feature when pressure abruptly rises immediately after full-eject occurs. The results of measurements of pump cycles of two different embodiments of the pump, performed according to this detection scheme, are illustrated in FIG. 9: curves P1, P2, and the respectively corresponding dP1/dt, dP2/dt clearly evidence the occurrence of the full eject in the first pump cycle and the lack of full-eject during the second pump cycle. The skilled artisan will immediately appreciate that the use of a time derivative of pressure is more sensitive and less prone to error than the use of pressure itself in determining if full-eject has occurred.

### EXAMPLE 2

In a related case (and considering for simplicity of presentation the case of the piston/cylinder compression mechanism, FIG. 6), the volume of the cylinder is coupled directly to the volume of the activating chamber 410 of the air-activated pump 400. During the relevant portion of the ejection stroke, the number of air molecules in this combined region does not change. Further, for a pump that operates isothermally (where the temperature of the air used to actuate the pump does not change) one can employ the ideal gas law to implement another embodiment of the invention.

As is well-known in the art, gasses generally conform to the ideal gas law,

$$P V = n R T , \qquad ( 1 )$$

where P represents the pressure of gas delivered to the activating chamber from the compression mechanism, V represents gas volume, n is the number of gas molecules, R is a known constant, and T is the gas temperature. Expressing the gas volume as the combination of the volume V_{ac} of gas in the activating chamber 410 of the pump-system 400 and the volume V_{cyl} of gas contained in the actuator (piston) cylinder and the passage connecting the actuator cylinder with the activating chamber at the very beginning of the systolic portion of the pump cycle, see FIG. 6 (that is, at the moment of time substantially corresponding to the outermost left point of the waveform 800A of FIG. 8A), one obtains

$$P {V}_{cyl} = n R T - P {V}_{a c} \qquad ( 2 )$$

The assumption here is that the user knows the volume of the actuator cylinder (and, in general, the relevant volume of the compression mechanism) but does not know the volume of the activating chamber (since the very problem being solved is to determine/identify the occurrence of the full-eject). Taking the time derivative of Eq. (2) results in

$$\frac{d}{dt} ( nRT - P {V}_{a c} ) = \frac{d}{dt} P {V}_{cyl} \qquad ( 3 )$$

Since nRT is assumed constant, then when the volume of the activating chamber becomes substantially static (~ does not change any more) due to all fluid 2 having been expelled from the activated chamber 412 (see FIG. 5B), the left-hand side of Eq. (3) equals to zero. That is, when full-eject occurs at the end of the systolic portion of the pump cycle,

$$\frac{d}{dt} P {V}_{cyl} = 0 \qquad ( 4 )$$

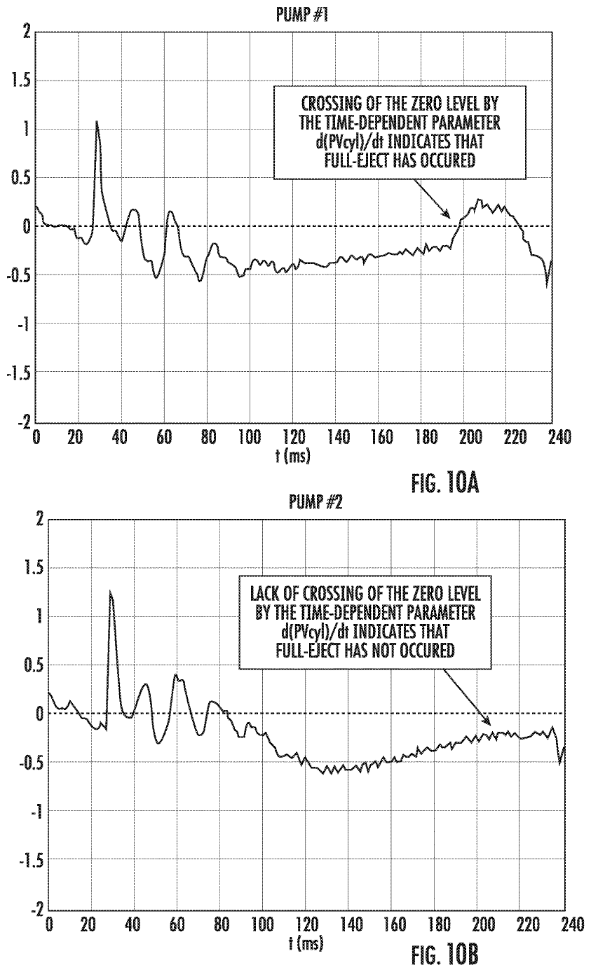

Using the same experimental data utilized in FIG. 10 of Example 1, one arrives at the results presented in FIGS. 10A and 10B.

$$Notably , \frac{d ( {PV}_{cyl} )}{dt} = 0 = \frac{1}{P {V}_{cyl}} \frac{d ( {PV}_{cyl} )}{dt} = \frac{d}{dt} ln ( {PV}_{cyl} ) \qquad ( 5 )$$

Accordingly, alternatively or in addition, the change in time of the natural log of the PV product measured at the compression mechanism on the side of the activating chamber to ascertain an occurrence of full-eject (or lack thereof), if there are practical advantages in doing so. (The skilled person will readily appreciate that the practical advantage of utilizing the natural logarithms based calculation presents itself when the electronic circuitry used to process these measurement data is of the type of analog electronic circuitry.)

The overall apparatus includes an embodiment of the pump system as discussed above, a pneumatic driver configured to provide pneumatic power to the pump system and containing a compression mechanism configured to reposition an activating fluid between a first fluid location and the activating chamber of the pump system, a gauge apparatus operably cooperated with the activating fluid, and electronic circuitry. Specifics of the implementation of the hardware of the embodiment of the invention differ depending, in part, on the type of the pneumatic driver. When operation of the compression mechanism turns on the use of piston/cylinder (FIG. 6), the first fluid location is that between the piston and the activating chamber. When operation of the compression mechanism of the pneumatic drive turns on the use of the source of compressed gas (FIGS. 7A, 7B), the first location is that between the in-line valve (providing access to the compressed gas from the source 230 to the chamber 41). The gauge apparatus is configured to measure at least a time-dependent value of pressure (produced by the activating fluid in the activating chamber during an operation of the pneumatic driver) and the volume of activating fluid delivered to the activating chamber, and may be appropriately structured as one of the many devices used for this purpose in the art.

The real-time determination of whether the operation of the pump system satisfies a full-eject condition can be carried out—at least in one case—by electronic circuitry operably cooperated with the gauge apparatus based on the measurement data received from the gauge apparatus. The same electronic circuitry mat be configured to produce an output signifying a result of such determination. (Optionally, such electronic circuitry includes analog circuitry and/or a digital circuitry such as a programmable processor.) Optionally, based on the determination whether the full-eject condition is achieved, the electronic circuitry may not only govern the operation of the overall apparatus (for example, to identify the pressure level at which the activating fluid should be delivered to the activating chamber from the pneumatic drive to ensure that the full-eject is observed in each of the pumping cycles) but also provide indicia of whether it is appropriate to replace a given pneumatic drive with another one (which may be the case when operational characteristics of the first pneumatic drive simply do not allow the apparatus to reach the full-eject during the pumping cycle, for example due to inability of the first pneumatic drive to create the pressure in the activating chamber that would lead to the full-eject).

For the purposes of this disclosure and accompanying claims, a real-time performance of a system is understood as performance that is subject to operational deadlines from a given event to a system's response to that event. For example, a real-time extraction of data (such as that representing a reading of the flow meter or a pressure sensor) from a buffer of a corresponding device or from tangible non-transient storage medium may be one triggered by the user or an appropriate electronic circuitry and executed substantially simultaneously with and without interruption of a measurement during which such data have been recorded.

As the skilled artisan will readily appreciate, one practically applicable way of improving the operation of the pump system once it is discovered that the full-ejection condition does not occur (as in the example of operation of pump #2 in FIG. 9 or pump #2 in FIG. 10B) would be implemented by slowly varying the duration of the systolic period of the pump cycle until the full-eject signal is detected. For example, the duration of the systolic portion of the pump cycle can be first slowly increased, then reduced and then increased again to regain the full-eject signal.

Depending on the specifics of the implementation of the hardware of the embodiment of the invention, the overall pneumatically-driven pump apparatus discussed thus far includes a pump system, a pneumatic driver configured to provide pneumatic power to the pump system, and electronic circuitry operably cooperated at least with a gauge apparatus of the pneumatic driver. The pump system has a rigid shell defining an internal volume that is separated by a reversibly movable chamber separator into an activating chamber and an activated chamber. The pneumatic driver includes: a compression mechanism configured to reversibly reposition an activating fluid between a first fluid location and the activating chamber, and the gauge apparatus operably cooperated with the activating fluid and configured to measure at least a time-dependent value of pressure produced by the activating fluid at the activating chamber during an operation of the pneumatic driver and to generate data representing results of such measurement. The electronic circuitry is configured to determine in real time, based at least in part on the data received from the gauge apparatus, whether a full-eject condition was satisfied during an operation of the pump system and to produce an output representing a result of such determination. In at least one implementation of the apparatus, at least one of the following conditions is satisfied: the chamber separator is configured as a flexible diaphragm that is substantially fluidly impenetrable; a portion of the compression mechanism containing the activating fluid is fluidly connected only with the activating chamber; and the electronic circuitry is configured to determine, as a function of time and based on the data, a value of a time derivative of the at least time-dependent value of pressure produced by the activating fluid at the activating chamber. Optionally, and substantially in every implementation of the apparatus, the compression mechanism may include a spatially-repositionable component. Optionally, and substantially in every implementation of the apparatus, the compression mechanism may incorporate (i) at least one piston slidably mounted inside a piston housing and/or (ii) a source of compressed fluid. Alternatively or in addition, in at least one of the embodiment the activated chamber includes an inlet and an outlet that may be spatially separated from one another. Alternatively or in addition, and substantially in every implementation of the apparatus, the electronic circuitry may include analog electronic circuitry and/or a processor configured to determine, based on the data, whether the pump system satisfies the full-eject condition by determining a time-dependent value of a time-derivative of a function of the at least the time-dependent value of the pressure. (In specific cases, such function may be a product of a volume of the activating fluid in the compression mechanism that is repositioned between the first fluid location and the activating chamber and the pressure—or a natural logarithm of such product.) Optionally, the pump system may be configured as a ventricle portion of an implantable artificial heart apparatus.

For the purposes of this disclosure and accompanying claims, a real-time performance of a system is understood as performance that is subject to operational deadlines from a given event to a system's response to that event. For example, a real-time extraction of data (such as that representing a reading of the flow meter or a pressure sensor) from a buffer of a corresponding device or from tangible non-transient storage medium may be one triggered by the user or an appropriate electronic circuitry and executed substantially simultaneously with and without interruption of a measurement during which such data have been recorded.

As the skilled artisan will readily appreciate, one practically applicable way of improving the operation of the pump system once it is discovered that the full-ejection condition does not occur (as in the example of operation of pump #2 in FIG. 9 or pump #2 in FIG. 10B) would be implemented by slowly varying the duration of the systolic period of the pump cycle until the full-eject signal is detected. For example, the duration of the systolic portion of the pump cycle can be first slowly increased, then reduced and then increased again to regain the full-eject signal.

Notably, according to idea of the invention, the above information can also be used to determine when full-eject happens (as compared to just ensuring that it does happen): the judicial cooperation of the appropriate sensor(s)/gauge(s) with the pump system 400 to measure n, T, P, and Vcyl during the operation of the pneumatic drive 420, 620 allows the user to rather precisely identify the point in time when the pump system satisfies the full-eject condition (as long as the full-eject occurs), see Eq. (3)

As a corollary of such determination, the user is also enabled to not only modify the pump cycle by ceasing the process of filling of the activating chamber with driving fluid 1 (air/gas) and starting the process of expelling the air/gas from the activating chamber and filling the activated chamber with fluid 2—thereby increasing the efficiency of operation of the pump system—but also to determine the volume of fluid 2 ejected from the activated chamber of the pump by the time the full-eject condition has been met. (Such volume may be referred to herein as a full-eject volume). To have either of these tasks carried out, an embodiment of the pump system 400 may be operably cooperated with and governed by an appropriate electronic circuitry (such as, in one specific case, a computer processor or, optionally, analog electronic circuitry) that is not shown in the drawings for simplicity of illustrations. For example, the electronic circuitry configured as a programmable processor may cause the pneumatic driver to reverse a flow of the activating fluid into the activating chamber (that is, to stop the systolic portion of the pump cycle and to initiate the diastolic portion of the pump cycle) substantially at the moment of time when the full-eject occurs (thereby at least reducing the overall length of the pump cycle and unnecessary wear of the pump and saving energy). Alternatively, the chosen electronic circuitry that performs the full-eject detection may be configured to control a vent valve that releases pressure from the driveline 426. That way the drive motor does not have to overcome the high pressure after full-eject takes place and the diastolic phase will automatically start sooner. (This alternative configuration assumes that a compression mechanism drive motor runs at a fixed rate and that elimination of the compression pressure is employed to reduce the work load on the motor and to initiate the diastolic portion of the pump cycle after full-eject takes place.) In other words, the idea of the invention stems from the realization that modification of the operation of a pneumatically-driven pump system can be made dependent on knowing at least some of the parameters of the fluid medium used to activate the operation of the pump system.

The need to be able to determine the full-eject volume arises from and is driven by a necessity to ensure that the fluid 2 flow delivered to the environment through the outlet is operationally sufficient for the application in which the pump system is employed. (In the specific case of the use of the pump system in an artificial heart apparatus, for example, it is critical that the pump provides a minimum of 3.5 liters per minute blood flow.) A conventional technique to ascertain the volume of fluid expelled (through the outlet, see FIG. 4) from the activated chamber 412 during the systolic portion of the pump cycle is to measure how much of fluid 2 enters the activated chamber 412 through the inlet during the diastolic portion of the pump cycle. This can be done using a mass flow sensor configured to measure the amount of fluid 1 (air/gas) exiting the air side of the of the pump 400 ventricle through the passage 426 as it is displaced by the incoming fluid 2. One then simply assumes that all that fluid 2 is ejected from the activated chamber 412 during systole. The skilled person would not question that, when such technique is employed, dependability and accuracy issues arise—specifically, when full-eject simply does not occur during the operation of the pump. In that case, the assumption that full-eject has occurred simply renders the determination of the ejected volume (of fluid 2) estimate completely unreliable. Such unreliability, coupled with the need for an additional flow sensor to carry out the required determination, understandably adds unnecessary cost and an additional point of failure in the pump system.

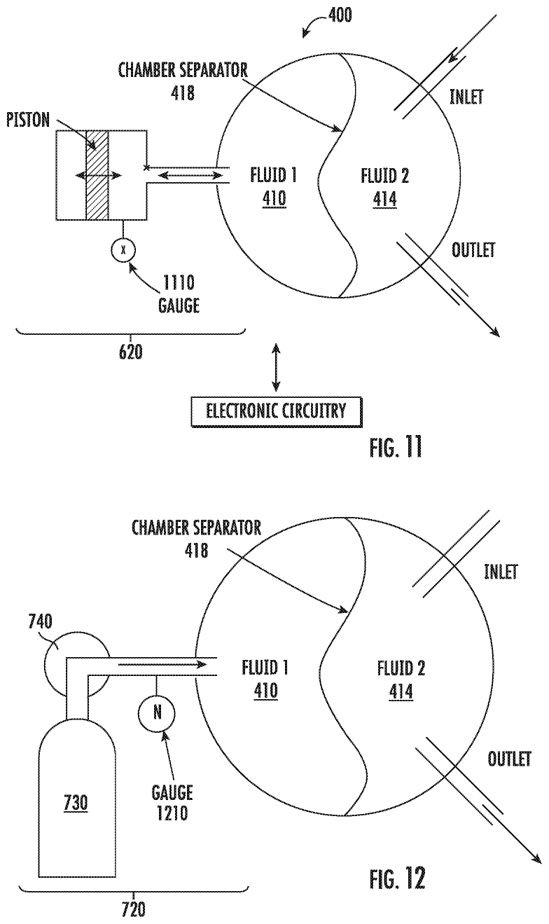

Embodiments of the invention provide a solution to this problem. Indeed, and referring again to Eq. (3), to compute the full-eject volume one needs to know (in addition to the value of pressure of gas delivered to the activating chamber from the compression mechanism of the pneumatic drive 412) either the volume of gas displaced by incoming into the activated chamber 412 fluid 2 during diastole, the number of moles of fluid/gas injected into the pump during systole, or the piston/cylinder volume change of the pneumatic drive 420 up to the point of full-eject (all of which are values that can be measured on the side of the pneumatic drive/activating chamber and that are referred to as “measured air-volume values”).
(A) In an example of the embodiment of the apparatus containing the pump system equipped, as shown in FIG. 11, with a volume-change type compression mechanism of FIG. 6, the following considerations are observed at least during the systolic portion of the pump cycle: (a) the number of gas molecules is fixed (dn/dt=0), that is cylinder of the compression mechanism is fluidly sealed, and (b) the change in piston/cylinder volume directly relates to the change in pressure and is slow enough. Then the pump system is maintained in approximately thermal equilibrium and the systolic portion of operation of the pump may be approximated as the isothermal compression (dT/dt=0) of fluid 1, and the general detection algorithm can be expressed, starting with Eq. (3) above, as

$$\frac{d}{dt} ( ln ( X ) + ln ( P ) ) = 0 , \qquad ( 6 )$$

where X represents a reading of the piston position gauge 1110 and P is the pressure at the activating chamber 410.
(B) In an alternative example of the embodiment of the pump system that utilizes, as shown in FIG. 12, a source 720 of the compressed fluid 1, since the overall volume on the activating side of the pump is simply the volume of the activating chamber 410, the general detection algorithm becomes

$$\frac{d}{dt} ( ln ( N ) + ln ( P ) - ln ( T ) ) = 0 , \qquad ( 7 )$$

where N represents a reading of the mass-flow sensor gauge 1210 and T and P represent, respectively, the temperature and pressure on the side of the activating chamber of the pump system.

Notably, a skilled person can determine that the overall apparatus does not operate in the isothermal regime but, for example, the adiabatic regime—and adjust the above-discussed formulae accordingly. Overall, the volume of fluid 2 ejected from the activated chamber 412 is substantially linearly related to these measured air-volume values (with appropriate linear constants that are determined by the dedicated experiment such as a calibration of the instrument). The moles of air can be determined from the mass air-flow sensor, while the piston/cylinder volume change can be determined with a piston position sensor or, as the skilled person will appreciate, from the angle change of the motor that is driving the piston. (In the specific example when the embodiment of the invention is utilized in an artificial heart apparatus, monitoring of the values of n, T, P, and V at the start of ejection, SOE, and the end of systole, EOS, allows accurate computation of blood flow per heartbeat. Monitoring heart rate then enables one to compute an average cardiac output in liters per minute, LPM.)

In at least one practical case, the full-eject volume could be determined using the full-eject feature or signal identified as discussed in reference to FIG. 9 applied to the pneumatic driver piston-driving motor angle position at the measured time of occurrence of the full-eject.

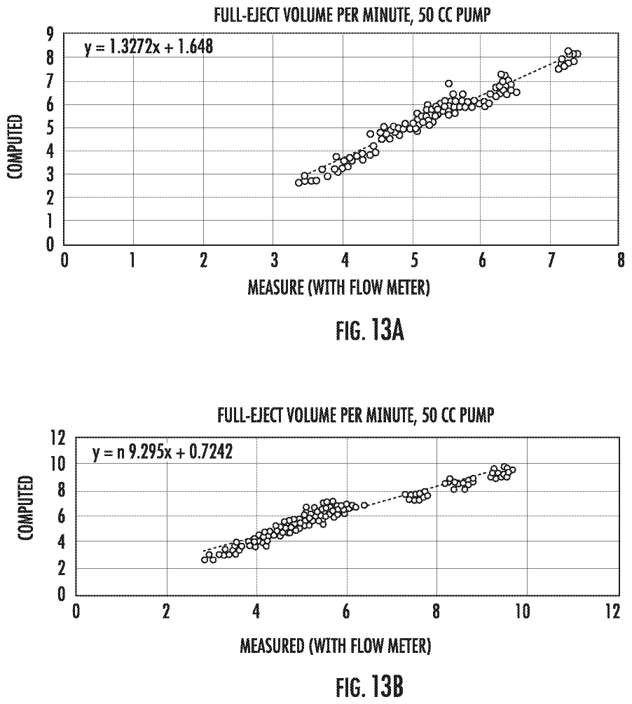

Typical empirical data, presented in plots of FIGS. 13A and 13B, illustrate the correspondence between the values of the measured full-eject volume of the fluid 2 out of the outlet of the activated chamber 412 and those computed as discussed above for air-operated pump systems of two different sizes (50 cubic centimeters and 70 cubic centimeters) using multiple pneumatic actuators under a wide variety of conditions. The measurements were performed with the use of a mock circulatory loop such as that provided by a well-known in the art Donovan Mock Circulation System (DMCS, discussed, in Donovan, F. M.,1975, Biomaterials, Med. Devices, Artif. Organs 3, 439-449. doi:10.3109/10731197509118635; the disclosure of which is incorporated by reference; or described elsewhere in related art), which is a tank system containing multiple tank chambers with appropriate resistance valves and/or flow meters or sensors through which fluid 2 flows after being ejected through the outlet of the pump system 400 and prior to being returned to the inlet of system 400. Tank chambers, therefore, are configured to simulate desired resistance to the flow of fluid 2. The test protocol includes recordation of various pressures in the tank chambers with the use of the DMCS monitoring software under various simulated conditions (such as variable flow rate of fluid 2 through the outlet and/or different rate of cyclical operation of the pneumatic drive 420), and comparing the results computed according to an embodiment of the invention. The substantial accuracy of the correspondence between the empirical and the computed dependencies attests to the legitimacy of the method and, therefore, the legitimacy of the full-eject detection technique described.

For the purposes of this disclosure and the appended claims, the expression of the type “element A and/or element B” is defined to have the meaning that is equivalent to “at least one of element A and element B”.

The use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

Within this specification, embodiments have been described in a way that enables a clear and concise specification to bet written, but it is intended and will be appreciated that embodiments may be variously combined or separated without parting from the scope of the invention. In particular, it will be appreciated that all features described herein at applicable to all aspects of the invention.

It is understood that, regardless of whether a particular reference to the use of a processor has been made in this disclosure, the implementation of the method of the invention may and preferably does include a processor controlled by instructions stored in a memory. The memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

While the invention is described through the above-described exemplary embodiments, it will be understood by those of ordinary skill in the art that modifications to, and variations of, the illustrated embodiments may be made without departing from the inventive concepts disclosed herein. Accordingly, the implementation of the invention should not be viewed as being limited to the disclosed embodiment(s).

1-23. (canceled)

24. An apparatus comprising:
a total artificial heart (TAH) system that includes at least one artificial ventricle configured to be coupled to at least one of a vessel of a heart and a chamber of the heart, the at least one artificial ventricle having an internal volume separated by a flexible and reversibly movable diaphragm into an activating chamber and an activated chamber;
a pneumatic driver configured to provide pneumatic power to the at least one artificial ventricle, wherein the pneumatic driver includes:
a compression mechanism configured to reversibly reposition an activating fluid between a first fluid location and the activating chamber; and
a gauge apparatus operably cooperated with said activating fluid and configured:
to carry out a measurement of
(A) at least a time-dependent value of a first pressure produced by the activating fluid at the activating chamber during an operation of the at least one artificial ventricle connected to a circulatory system of a body, and
(B) (i) a time-dependent value of a change in a volume of the compression mechanism against a second pressure produced by the circulatory system in the activated chamber;
or
(ii) a number of moles of the activating fluid delivered to the activating chamber during a systolic portion of a pump cycle and temperature at the activating chamber, and
to generate data representing results of the measurement;
and
electronic circuitry operably cooperated with the gauge apparatus and configured:
(a) to cease repositioning of the activating fluid towards the activating chamber substantially at a moment of time when an operation of the at least one artificial ventricle connected to the circulatory system satisfies a full-eject condition;
(b) to determine in real time and based at least in part on the data received from the gauge apparatus
a change of a volume of the activated fluid in the activated chamber of the at least one artificial ventricle connected to the circulatory system that occurs between a beginning of the systolic portion of a pump cycle of the at least one ventricle and said moment of time;
and
(c) to produce a user perceivable output that represents a result of corresponding determination,
wherein the apparatus is configured to satisfy the full-eject condition at the at least one artificial ventricle of the TAH system in operation thereof.

25. An apparatus according to claim 24,
wherein the compression mechanism includes a piston repositionable in a piston housing, and the gauge apparatus is configured to identify, as a function of time, a position of the piston within the piston housing;
or
wherein the compression mechanism includes a source of compressed activating fluid, and the gauge apparatus includes a mass-flow sensor gauge and a device configured to measure temperature.

26. An apparatus according to claim 24, wherein at least one of the following conditions is satisfied:
the chamber separator is configured as a flexible diaphragm that is substantially fluidly impenetrable; and
a portion of the compression mechanism containing said activating fluid is fluidly connected only with the activating chamber; and
the electronic circuitry is configured to determine, as a function of time and based on said data, a value of a time derivative of the at least time-dependent value of the first pressure produced by the activating fluid at the activating chamber.

27. An apparatus according to claim 24, wherein one of the following conditions is satisfied:
the electronic circuitry is configured to activate a vent valve cooperated with a driveline that fluidly connects the activating chamber with the pneumatic driver to release pressure from the driveline to initiate a diastolic portion of the pump cycle substantially at the moment of time when the operation of the at least one artificial ventricle connected to the circulatory system satisfies the full-eject condition, and
the electronic circuitry is configured as a processor programmed to cause the pneumatic driver to reverse a flow of the activating fluid between the first location and the activating chamber substantially at said moment of time when the operation of the at least one artificial ventricle connected to the circulatory system satisfies the full-eject condition.

28. An apparatus according to claim 24, wherein the compression mechanism includes a spatially-repositionable component.

29. An apparatus according to claim 24,
wherein the compression mechanism incorporates (i) at least one piston slidably mounted inside a housing and/or (ii) a source of compressed fluid.

30. An apparatus according to claim 24, whether the activated chamber includes
a) a tricuspid valve of the TAH system and a pulmonary valve of the TAH system; or
b) a bicuspid valve of the TAH system and an aortic valve of the TAH system.

31. An apparatus according to claim 24,
wherein the electronic circuitry is configured to determine whether the at least one artificial ventricle satisfies the full-eject condition at the activated chamber by determining a time-dependent value of a time-derivative of a function of at least the time-dependent value of said first pressure.

32. An apparatus according to claim 31, wherein the function is:
(i) a product of a first volume and a first pressure, wherein the first volume is a volume of the activating fluid in the compression mechanism being repositioned between the first fluid location and the activating chamber; or
(ii) a natural logarithm of such product.

33. An apparatus according to claim 24, further comprising:
a tangible non-transitory computer readable medium, operably coupled with the electronic circuitry and containing:
(1) program code for determining said moment of time when the operation of the at least one artificial ventricle connected to the circulatory system satisfies the full-eject condition by solving an equation in which a time-derivative of a sum of
(i) a first function of a reading of the mass-flow sensor gauge connected to the activating chamber,
(ii) a second function of a time-dependent value of said first pressure, and
(iii) a negative of a third function of the temperature at the activating chamber
is substantially equal to zero;
and/or
(2) program code for determining said moment of time when the operation of the at least one artificial ventricle connected to the circulatory system satisfies the full-eject condition by solving an equation in which a time-derivative of a sum of
(a) a fourth function of a reading of a piston position gauge of the gauge apparatus and
(b) the second function of the time-dependent value of the first pressure is substantially equal to zero.

34. An apparatus according to claim 30, wherein every of the first function, the second function, the third function, and the fourth function is a natural logarithm to enable use of the electronic circuitry that includes analog electronic circuitry.

35. A method comprising:
with the use of electronic circuitry:
recording, in time, a first time-dependent function of at least a value of a first pressure produced by the activating fluid in the activating chamber of the at least one ventricle of the TAH system of the apparatus according to claim 24 during repositioning of the activating fluid between the compression mechanism and the activating chamber of the at least one ventricle of the TAH system and measured with the gauge apparatus;
identifying at least one of (i) a temperature of the activating fluid, a parameter related to a volume occupied by the activating fluid, a mass flow rate of the activating fluid between the first fluid location and the activating chamber, and (ii) a position of a spatially-repositionable component of the compressing mechanism of the apparatus.

36. A method according to claim 35, further comprising, with the use of the electronic circuitry and with the use of results of said recording and identifying, calculating at least a moment of time when the at least one ventricle of the TAH system satisfies a full-eject condition during operation of the TAH system.

37. A method according to claim 35, further comprising, with the use of the electronic circuitry, determining a full-eject volume characterizing the operation of the at least one ventricle of the TAH system.

38. A method according to claim 35, wherein said recording the first time-dependent function includes:
a) recording, with a computer processor, a time derivative of the at least said value of the first pressure modified by the parameter; or
b) recording, with analog electronic circuitry, a time derivative of a natural logarithm of the at least said value of the first pressure modified by the parameter.

39. A method comprising:
controlling a functioning of the total artificial heart according to claim 24 by at least:
limiting the first pressure produced by the activating fluid at the activating chamber during an operation of the at least one artificial ventricle connected to a circulatory system of a body at a level defined by pressure reached at the activating chamber at the moment of time when an operation of the at least one artificial ventricle satisfied a full-eject condition.

40. A method according to claim 39, wherein the limiting includes:
with the use of said electronic circuitry,
(i) activating a vent valve cooperated with a driveline that fluidly connects the activating chamber with the pneumatic driver to release pressure from the driveline to initiate a diastolic portion of the pump cycle substantially at said moment of time, wherein the compression mechanism includes a source of compressed fluid; or
(ii) causing the pneumatic driver to reverse a flow of the activating fluid between the first location and the activating chamber substantially at said moment of time, wherein the compression mechanism includes a piston repositionable within a piston housing.

41. A method according to claim 39, further comprising: maintaining fluidic pressure provided by the pneumatic driver to the activating chamber during a systolic portion of a pump cycle to maintain a full-eject volume from an activated chamber of the at least one artificial ventricle substantially constant from one pump cycle to another pump cycle.

42. A method according to claim 39, comprising: measuring a moment of time when the operation of the at least one ventricle satisfies the full-eject condition.

43. A computer program product for governing an operation of a pneumatically driven artificial ventricle of a total artificial heart (TAH) system coupled to at least one of a vessel of a heart and a chamber of the heart,
the computer program product comprising a computer usable tangible non-transitory medium having computer readable program code thereon, the computer readable program including:
program code for determining a moment of time when the operation of the artificial ventricle, operably fluidly connected with a pneumatic driver, satisfies a full-eject condition; and
program code for ascertaining a full-eject volume characterizing the operation of said artificial ventricle of the TAH system.

44. A computer program product according to claim 43, wherein said program code for determining includes program code for determining the moment of time based on a product of a value of pressure of activating fluid delivered from the pneumatic driver to an activating chamber of the artificial ventricle and a value representing a volume of the activating fluid repositioned by the pneumatic driver to the activating chamber.

45. A computer program product according to claim 43, wherein said program code for ascertaining includes:
(i) program code for defining a sum of a first function of a reading of a piston position gauge of a gauge apparatus of the pneumatically driven artificial ventricle of the TAH system and a second function of a pressure of activating fluid produced by the pneumatic driver at an activating chamber of the artificial ventricle;
and/or
(ii) program code for defining a sum of the second function, a third function of a reading of a mass-flow sensor gauge of the gauge apparatus, and a fourth function of a temperature at the activating chamber.
